(19) United States
(12) Patent Application Publication
(10) Pub. No.: US 2014/0365170 A1
(43) Pub. Date: Dec. 11, 2014

(54) GRAIN CART WITH INTERGRATED MOISTURE SENSOR

(71) Applicant: Unverferth Manufacturing Company, Inc., Kalida, OH (US)

(72) Inventor: Micheal D. VAN MILL, Shell Rock, IA (US)

(73) Assignee: UNVERFERTH MANUFACTURING COMPANY, INC., Kalida, OH (US)

(21) Appl. No.: 14/466,591

(22) Filed: Aug. 22, 2014

Related U.S. Application Data
(63) Continuation-in-part of application No. 14/213,246, filed on Mar. 14, 2014.
(60) Provisional application No. 61/799,957, filed on Mar. 15, 2013.

(51) Int. Cl. A01D 90/12; G01N 19/10; G01N 33/24; A01D 90/10

(52) U.S. Cl. CPC A01D 90/12; A01D 90/10; G01N 19/10; G01N 33/24; G01N 2033/245
USPC 702/173; 414/507; 414/526; 414/21; 414/345

(57) ABSTRACT

A grain cart including a hopper for transporting harvested material. The hopper can include a first opening for receiving harvested material discharged from a harvesting vehicle, a second opening for unloading harvested material from the hopper, and a conveyor assembly operatively connected to the second opening so as to allow harvested material to be unloaded from the hopper using the conveyor assembly. The conveyor assembly can include a sump operatively connected to the second opening of the hopper so as to receive harvested material from the hopper, a conveyor housing including a discharge opening for discharging harvested material from the conveyor assembly, and a conveyor operatively connected to the sump so as to move harvested material from the sump to the discharge opening of the conveyor housing. The sump can include a moisture sensor designed to detect the moisture content of harvested material passed through the sump.

### CROSS-REFERENCE TO RELATED APPLICATIONS

This application is a continuation-in-part of U.S. application Ser. No. 14/213,246, filed Mar. 14, 2014, now pending, which claims the benefit of U.S. Provisional Application No. 61/799,957, filed on Mar. 15, 2013. The disclosures of these applications are incorporated herein in their entirety.

### BACKGROUND

1. Field

Embodiments of the present invention relate generally to grain carts, and more particularly, to grain carts including an integrated moisture sensor for detecting the moisture content of grain in the cart.

2. Background

Carts, such as grain carts, are used to shorten harvesting time by improving the efficiency of harvesting equipment such as combines. Such carts can, for example, be used to transport grain from harvesters or combines in the field to grain trucks or hoppers at the side of the field. Carts are often preferred for use compared to grain hoppers or grain trucks because grain hoppers are typically immobile and grain trucks typically do not perform well in muddy or rough field conditions and have the potential to spark fires in dry fields. Carts usually comprise a hopper (i.e., hopper or box) sitting atop a wheeled frame in combination with an auger means or mechanism for unloading grain from the hopper. Carts can be designed to handle soft or rough fields with ease and can be designed to be drawn by a tractor alongside a combine that unloads its contents into the cart. Carts can be used to enable a combine to continue to harvest while unloading the grain into the cart. This grain unloading arrangement can increase productivity dramatically because combines need never stop to unload. In addition, it is not necessary for the combines themselves to travel to grain trucks or hoppers at the side of the field each time the combine is full. After a cart is loaded with grain or other material by one or more combines, the grain is unloaded from the cart into a hopper for temporary storage or into a waiting grain truck for transport to another location, such as a grain elevator. Because carts offer a combination of economy, versatility, production savings, and maneuverability, they have been widely accepted by farmers and widely produced by equipment manufacturers.

Carts capable of unloading grain directly into a grain truck or hopper often use a conveyor to do so. Often the conveyor is in the form of an auger. Various auger configurations are known. Auger structures can, for example, be contained inside the hopper structure, located entirely outside of it, or in another desired location. Auger configurations can, for example, have a single auger or multiple augers. The auger structure can, for example, be located at the front, side, back, corner, or another desired location of the cart.

To control and monitor mobile farm implements such as grain carts and tractors, various sensors and controllers have been placed in the mobile farm implements to collect data or carry out commands. On some mobile farm implements, the sensors and controllers have been wired to connect to display terminals mounted in the mobile farm implements. There is a continuous need for improved grain carts and improved sensors and controllers for such carts.

### SUMMARY

Grain carts with integrated moisture sensors are presented. Because harvested material, such as grain, is often stored at lower moisture levels in order to prevent spoiling, it is not uncommon to harvest the grain at a higher moisture level and then run the higher moisture level grain through a drier to remove water. After the grain is dried down, the weight of the grain will have changed. By having a moisture sensor integrated in the grain cart itself to read the moisture level of the grain, the system can calculate the dry weight equivalent by formula so a user does not have to. Another advantage of including a moisture sensor in the grain cart itself is that instead of measuring the moisture sensor in each harvesting vehicle before the harvested material is provided to the grain cart, only a single moisture sensor is required.

According to some embodiments, a grain cart can include a hopper for transporting harvested material. The hopper can include a first opening for receiving harvested material discharged from a harvesting vehicle, a second opening for unloading harvested material from the hopper, and a conveyor assembly operatively connected to the second opening so as to allow harvested material to be unloaded from the hopper using the conveyor assembly. The conveyor assembly can include a sump operatively connected to the second opening of the hopper so as to receive harvested material from the hopper, a conveyor housing including a discharge opening for discharging harvested material from the conveyor assembly, and a conveyor operatively connected to the sump so as to move harvested material from the sump to the discharge opening of the conveyor housing. The sump can include a moisture sensor designed to detect the moisture content of harvested material passed through the sump.

According to some embodiments, a method for calculating a dry weight of harvested material discharged from a grain cart can include discharging harvested material from a harvest vehicle into a hopper of the grain cart, receiving harvested material in a sump connected to an opening of the hopper, passing the harvested material through the sump to a discharge opening of a conveyor housing so as to discharge harvested material from the grain cart, wherein the step of passing the harvested material through the sump includes passing the harvested material past a moisture sensor disposed within the sump that is designed to detect the moisture content of harvested material passed through the sump, and calculating a dry weight of harvested material received within the hopper based on the moisture content of harvested material passed through the sump and the weight of the harvested material received in the hopper.

Other features and advantages of embodiments of the invention will become apparent to those of skill in the art upon reviewing the following detailed description of the preferred embodiments and the accompanying drawings.

### BRIEF DESCRIPTION OF THE DRAWINGS

The accompanying drawings, which are incorporated herein and form part of the specification, illustrate embodiments and, together with the detailed description, serve to explain the principles of the invention and to enable a person skilled in the art to make and use the invention. In the drawings, like reference numbers are used to indicate identical or functionally similar elements.

FIG. 1 illustrates a side perspective view of a grain cart in accordance with an embodiment.

FIG. 2 illustrates an enlarged cut-away view of section A of FIG. 1.

FIG. 3 illustrates an exploded view of a door assembly of the grain cart of FIG. 1.

FIG. 4 is a block diagram of a control unit for a moisture sensor assembly of the grain cart of FIG. 1.

FIG. 5 illustrates a side view of a sump of the grain cart of FIG. 1.

FIG. 6 illustrates a cross-sectional view of the sump of FIG. 5 along a rotational axis of a conveyor of the sump.

FIG. 7 illustrates a side perspective view of a grain cart in accordance with another embodiment.

FIG. 8 illustrates an enlarged cut-away view of section B of the grain cart of FIG. 7.

FIG. 9 illustrates an exploded view of a door assembly of the grain cart of FIG. 7.

FIG. 10 illustrates a side view of a sump of the grain cart of FIG. 7.

FIG. 11 illustrates a cross-sectional view of the sump of FIG. 7 along a rotational axis of a conveyor of the sump.

FIG. 12 illustrates a method for calculating a dry weight of harvested material discharged from a grain cart.

### DETAILED DESCRIPTION OF THE PRESENT INVENTION

While the present invention may be embodied in many different forms, a number of illustrative embodiments are described herein with the understanding that the present disclosure is to be considered as providing examples and not intended to limit the invention to the preferred embodiments described and/or illustrated herein.

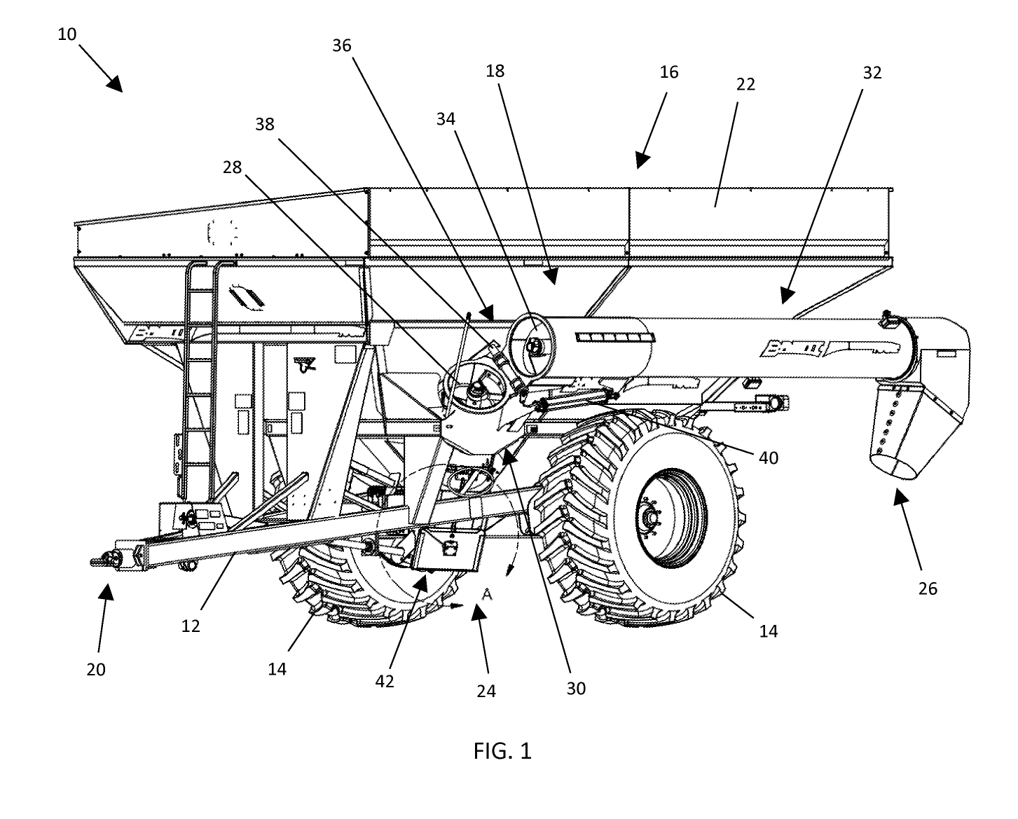

FIG. 1 illustrates a side perspective view of a grain cart 10 in accordance with an embodiment. Although the widely-known term “grain cart” is used herein, it is appreciated that such carts are often used to receive, transport, and store other agricultural materials received from a harvesting vehicle, such as for example seeds, wheat, canola, barley, soybeans or corn. The harvesting vehicle can, for example, be a combine harvester. Cart 10 can, for example, be designed to be pulled by a tractor or other suitable vehicle. In some embodiments, cart 10 can be self-propelled. As described further below, cart 10 can include various components, such as a frame 12, wheels 14, a hopper 16, and a conveyor assembly 18.

Frame 12 can, for example, be designed to support the various components of cart 10. In some embodiments of grain carts, such as cart 10 depicted in FIG. 1, frame 12 can include a hitch 20 at a front end for coupling cart 10 to a tractor or other vehicle to allow cart 10 to be towed. In some embodiments, frame 12 can house one or more mechanical linkages, such as for example a power-take off device for transferring mechanical power from a towing vehicle. Frame 12 can be a distinct and separate piece from hopper 16 and conveyor assembly 18 or can be formed from a single monolithic piece of material with these components.

Wheels 14 can, for example, be provided to assist in conveying cart 10 along a ground surface. As depicted in FIG. 1, cart 10 can include two wheels 14 disposed on each side of cart 10. It is appreciated that cart 10 can include fewer than two wheels 14 or more than two wheels 14 if desired. In some embodiments, cart 10 can include a suitable track or other form of conveyance instead of or in addition to wheels 14.

Hopper 16 can, for example be designed to receive, transport, and store grain, seed, corn, or other suitable harvested materials. Hopper 16 is defined at least partially by a plurality of hopper walls 22 that form a storage space converging at the opening of a sump 24 near a bottom of cart 10. Hopper 16 and hopper walls 22 can, for example, be oriented so as to funnel such material into a sump 24. For example, hopper 16 can include a first opening at the top of hopper 16 for receiving grain discharged from a harvesting vehicle and a second opening at the bottom of hopper 16 for unloading grain from hopper 16 into sump 24. The design of hopper walls 22 can provide for smooth flowing curves to allow the harvested material to be easily unloaded via conveyor assembly 18. This can help prevent the harvested material from sticking to the hopper walls 22, which can reduce the likelihood of corrosion of hopper walls 22. Hopper 16 can further include an integrated scale or other weighing unit for measuring a total weight of grain received within hopper 16. Hopper 16 can be sized so as to receive from about 12 to about 60 tons of harvested material. Other suitable hopper sizes can be used.

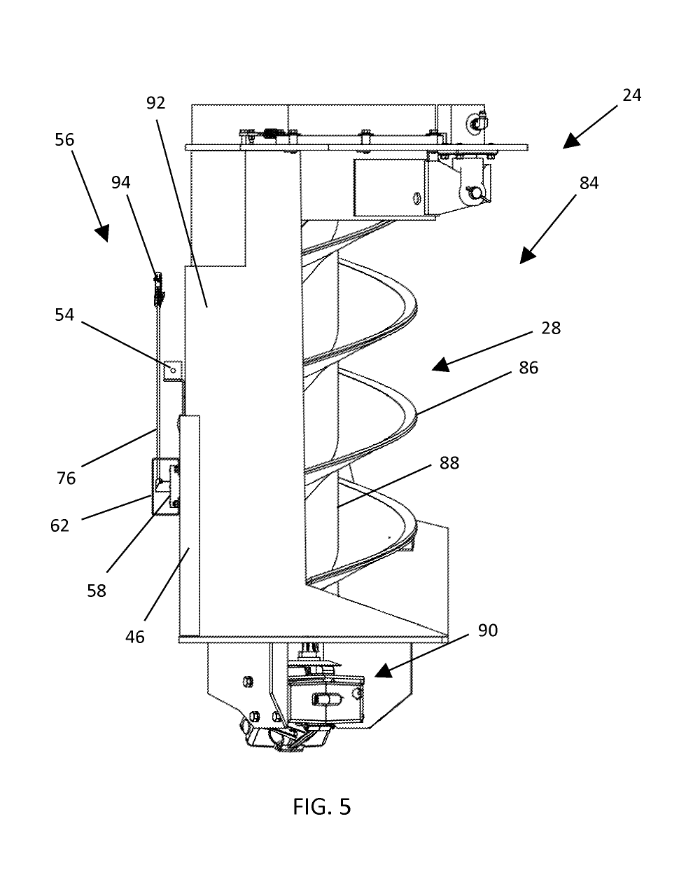

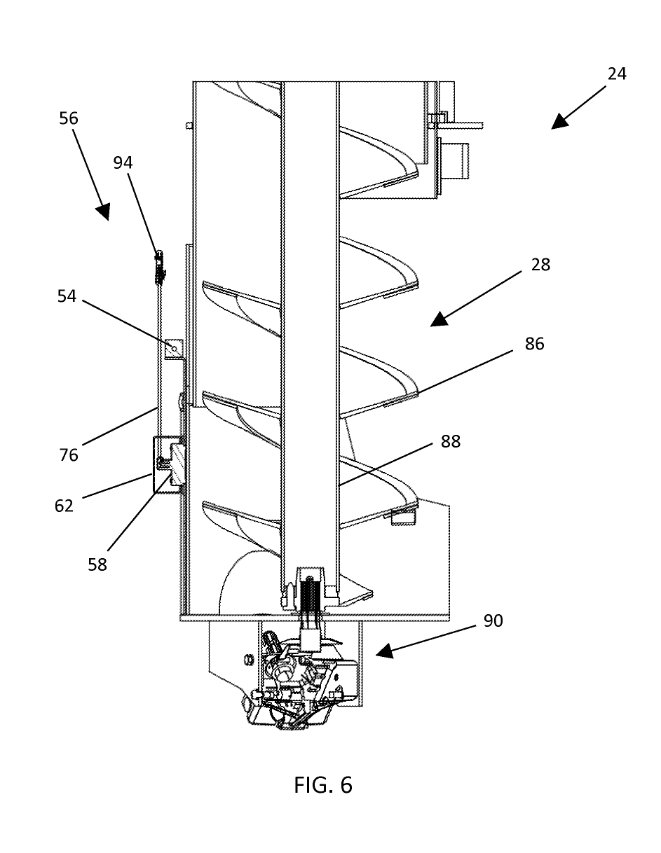

Conveyor assembly 18 can be operatively connected to sump 24 at the second opening of hopper 16 and is designed to convey harvested material from hopper 16 to a semi-trailer or other suitable moving or stationary receptacle. Harvested material is conveyed through a substantially tubular housing of conveyor assembly 18. Conveyor assembly 18 includes a discharge opening 26 at a discharge end thereof for discharging harvested material therefrom. As shown in further detail for example in FIGS. 5-6, in some embodiments, conveyor assembly 18 includes a lower conveyor 28, such as an auger, that extends into sump 24 to move grain or other material from sump 24 through conveyor assembly 18.

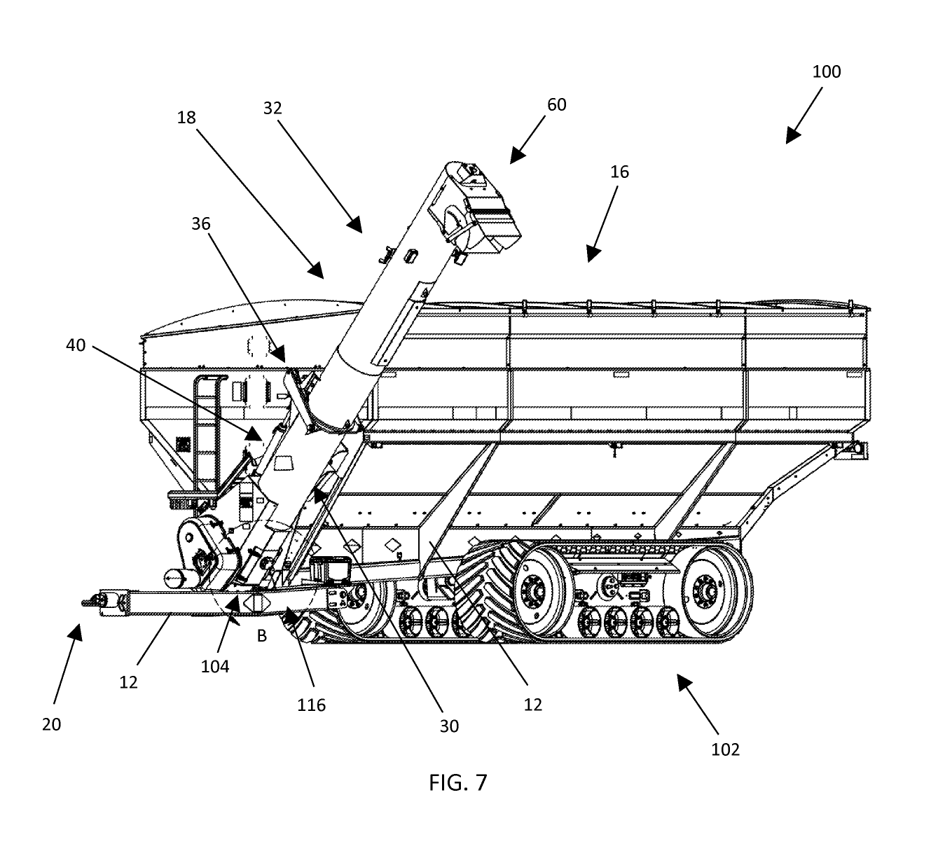

Conveyor assembly 18 is connected to and extends upwardly from sump 24 along a corner of hopper 16. Conveyor assembly 18 can include a lower conveyor housing 30 that houses lower conveyor 28 as well as an upper conveyor housing 32 that houses an upper conveyor 34. Upper conveyor housing 32 is coupled to lower conveyor housing 30 via a folding assembly 36. Folding assembly 36 is configured to allow an end of lower conveyor housing 30 to abut against a corresponding end of upper conveyor housing 32 in an unfolded/extended position, and further allows upper conveyor housing 32 to pivot about a folding joint 38 of folding assembly 36 (see, e.g., FIG. 1) relative to lower conveyor housing 30 to position conveyor assembly 18 in a folded position. In the folded position, upper conveyor housing 32 may be disposed along the outer surface of hopper 16 in a substantially horizontal direction. One example of such a folded position is depicted in FIG. 1. The folded position can, for example, be designed for use while cart 10 is in storage, transport, or in other situations. An illustration of a similar embodiment of a grain cart with its conveyor assembly in an unfolded/extended position is depicted in FIG. 7.

Folding assembly 36 can include a conveyor fold actuator 40 for assisting moving conveyor assembly 18 into its folded position. Fold actuator 40 can be rotatably fixed at a first end to lower conveyor housing 30 and rotatably fixed at a second end to upper conveyor housing 32. In some embodiments, the first end of fold actuator 40 can be fixed at a location other than lower conveyor housing 30 and the second end of fold actuator 40 can be fixed at a location other than upper conveyor housing 32. For example, in some embodiments, the first end of fold actuator 40 can be rotatably fixed on frame 12 with the second end fixed to upper conveyor housing 32. Conveyor fold actuator 40 can, for example, be a hydraulic cylinder designed to facilitate folding and unfolding of upper conveyor housing 32. Conveyor fold actuator 40 can be configured to lock upper conveyor housing 32 in either an unfolded or folded state as desired. When lower conveyor housing 30 and upper conveyor housing 32 are in the unfolded state, the two housings can form a single joint conveyor housing. Lower conveyor 28 can be removably coupled to upper conveyor 34 so as to provide a desired mechanical power transfer between lower conveyor 28 and upper conveyor 34 such that rotation of lower conveyor 28 causes a corresponding rotation of upper conveyor 34. In some embodiments, a lower end of upper conveyor 34 is generally longitudinally aligned with folding joint 38.

Lower conveyor 28 can, as shown for example in FIGS. 5-6, be in the form of an auger. Upper conveyor 34 can likewise be in the form of an auger or another suitable conveyor. When conveyor assembly 18 is in its extended position, lower conveyor 28 and upper conveyor 34 form a single conveyor that can be controlled from a lower end of the conveyor to move harvested material through conveyor assembly 18 and out of conveyor assembly 18 via discharge opening 26. In some embodiments, lower conveyor 28 and/or upper conveyor 34 can be in the form of another suitable conveyor type for a grain cart, such as a suitable belt conveyor. In some embodiments, a single conveyor (instead of two conveyors) extends through both lower conveyor housing 30 and upper conveyor housing 32. In some embodiments, conveyor assembly 18 is a single housing structure and is not able to fold between a folded position and an extended position. In one such embodiment, conveyor assembly 18 is fixed in its extended position.

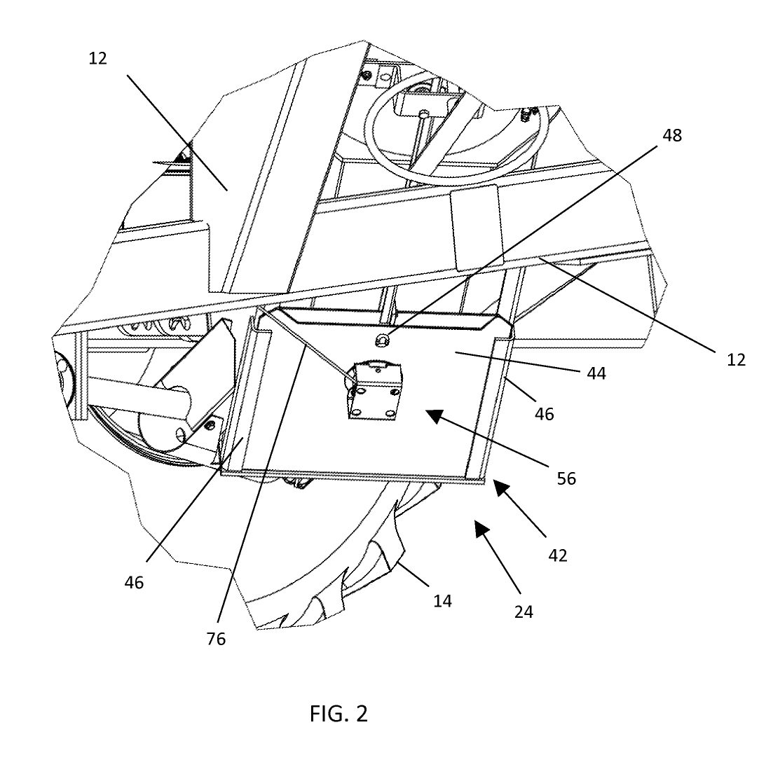

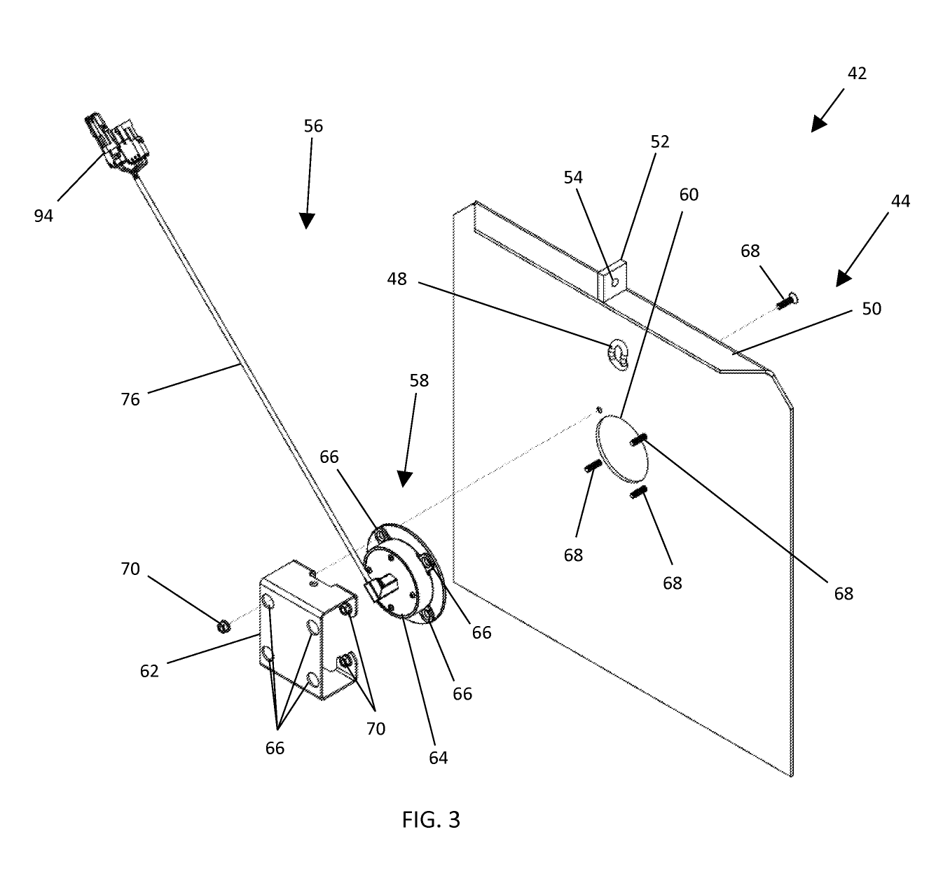

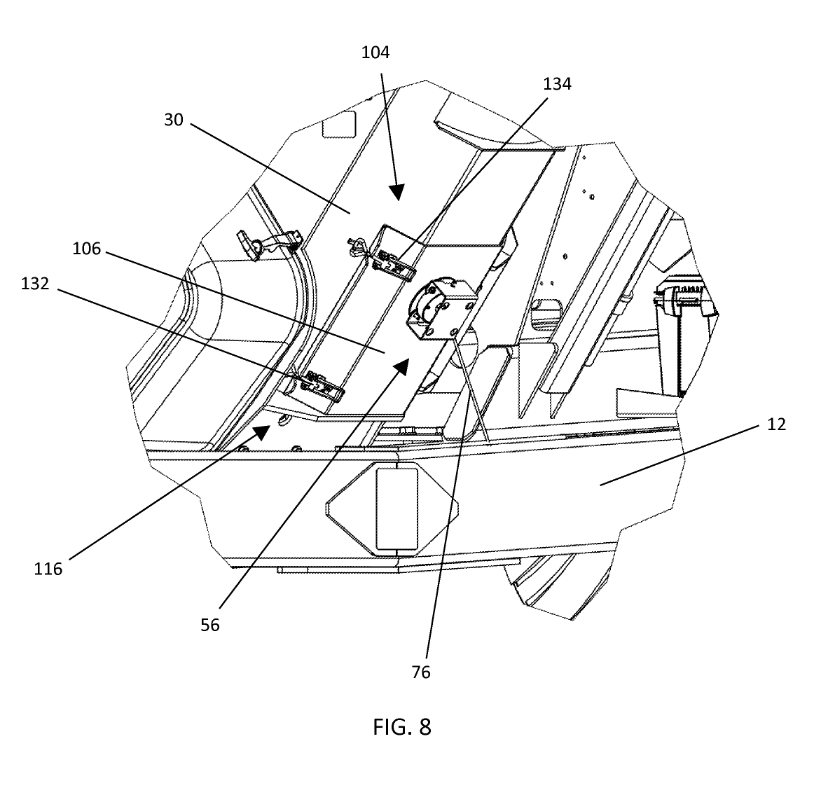

FIGS. 2 and 3 illustrate further details of sump 24 as well as a door assembly 42 of sump 24 that allows a user to access an interior of sump 24. In particular, FIG. 2 illustrates an enlarged cut-away view of section A of FIG. 1 that shows details of door assembly 42, and FIG. 3 illustrates an exploded view of door assembly 42. Door assembly 42 can be in the form of a completely removable (should for example in FIG. 3) or hinged (shown for example in FIG. 8) clean-out door that allows access to an interior cavity of sump 24. As illustrated in FIG. 3 for example, door assembly 42 can include a door panel 44 that is removably fixed to cart 10 via one or more slide rails 46 or other suitable structures. A finger grip 48 or other structure can be provided on door panel 44 to facilitate removal of door panel 44. In an embodiment, a rack 49 may be attached to the door panel 44, and a wheel 51 may engage the rack 49 such that rotation of the wheel 51 moves the door panel 44 between open and closed positions. Door panel 44 can further include a curved portion 50 that curves away from sump 24 and supports a projection 52 including a lockout hole 54 that can be aligned with a corresponding lockout hole on another portion of cart 10, such as frame 12. An operator can lock door panel 44 in place by passing a lock through the lockout hole 54 when it is aligned with its corresponding lockout hole. It is appreciated that other suitable locking structures can be used in order to securely lock door assembly 42 in place.

Door assembly 42 can further include an moisture sensor assembly 56 including a moisture sensor 58 for detecting the moisture content of grain passed through sump 24. Moisture sensor 58 can be in the form of a capacitive or other suitable sensor for detecting moisture in the harvested material. Moisture sensor 58 can be received within a corresponding opening 60 formed in door panel 44. Moisture sensor 58 can, for example, be aligned with opening 60 such that harvested material that passes through sump 24 is passed by moisture sensor 58 so as to allow moisture sensor 58 to detect the moisture content of the harvested material. In an embodiment of the present invention, the moisture sensor 58 may be positioned at a bottom of the sump 24 such that the grain and moisture passing through the sump 24 moves towards the sensor 58 due to gravity. In some embodiments, moisture sensor 58 has a sensing area with a diameter ranging from approximately 2.5 to approximately 3 inches. In some embodiments, moisture sensor 58 is recessed from an interior surface of door panel 44 facing sump 24. In some embodiments, moisture sensor 58 is flush with an interior surface of door panel 44 facing sump 24. In embodiments in which the grain cart includes a double auger, the moisture sensor 58 may be positioned at the sump of the first auger or at the junction box where the second auger receives material from the first auger.

Moisture sensor assembly 56 further includes a bracket 62 for fixing moisture sensor 58 to door panel 44. Bracket 62 can be designed to protect moisture sensor 58 from damage. For example, in some embodiments, bracket 62 can wrap around a housing 64 of moisture sensor 58. Door panel 44, housing 64, and bracket 62 can each include corresponding bolt holes 66 designed to receive bolts 68 for securing moisture sensor 58 to door panel 44. Bolts 68 can be secured in place via nuts 70 or other suitable structures.

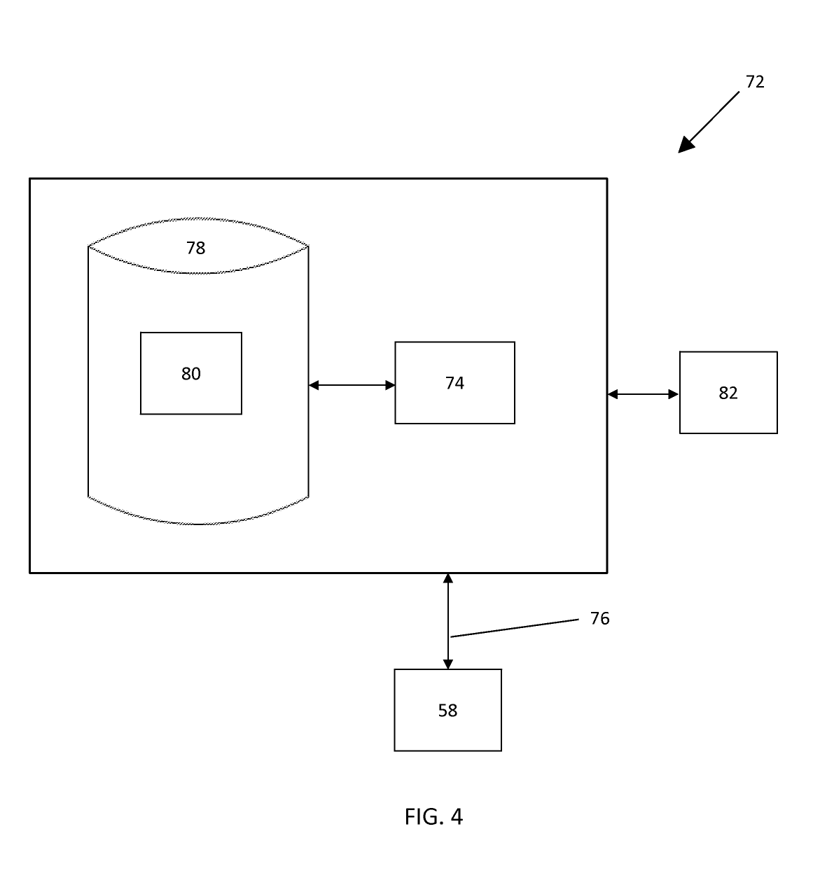

FIG. 4 is a block diagram of a control unit 72 for moisture sensor assembly 56. Moisture sensor 58 is operatively coupled to a processor 74 of control unit 72 via wiring 76 and an electrical connector 94. Processor 74 is operatively coupled to a data storage system 78, which can for example include one or more non-volatile storage devices and/or one or more volatile storage devices (e.g., random access memory (RAM)). Computer readable program code (CRPC) 80 may be stored in a non-transitory computer readable medium, such as, but not limited, to magnetic media (e.g., a hard disk), optical media (e.g., a DVD), memory devices (e.g., random access memory), and the like. In some embodiments, computer readable program code 80 is configured such that when executed by processor 74, the code causes processor 74 to perform steps described herein (e.g., steps described above with reference to one or more flow charts provided herein). In some embodiments, control unit 72 is configured to perform steps described herein without the need for code. For example, processor 74 may consist merely of one or more ASICs. Hence, the features of the embodiments described herein may be implemented in hardware and/or software. For example, in particular embodiments, the functional components of control unit 72 described herein may be implemented by processor 74 executing computer instructions, by processor 74 operating independent of any computer instructions, or by any suitable combination of hardware and/or software.

Processor 74 can, for example, be configured to calculate a dry weight of harvested material received within hopper 16 based on the moisture content of the harvested material passed through sump 24 and a weight of the harvested material received in hopper 16. In some embodiments, processor 74 is configured to calculate a dry weight of grain received within hopper 16 based on a predetermined crop standard moisture for grain within hopper 16. Cart 10 can further include an input device 82 operatively connected to processor 74 that allows an operator to select a type of grain received in hopper 16 to allow processor 74 to automatically calculate the dry weight of grain received within hopper 16. In some embodiments, control unit 72 can further include an output device that is integrated in input device 82 or as a standalone unit, such as a monitor display. Input device 82 can be in the form of a laptop, keyboard, touchscreen computer, such as a tablet, or another suitable input device.

FIGS. 5 and 6 illustrate views of a portion of sump 24 with certain elements removed to show details of sump 24. In particular, FIG. 5 illustrates a top view of sump 24 and FIG. 6 illustrates a cross-sectional view of sump 24 along a rotational axis of conveyor 28. Sump 24 includes a receiving area 84 that receives harvested material passed through hopper 16. As shown in these figures, in some embodiments, conveyor 28 is at least partially located in sump 24 and is in the form of an auger having a helical screw blade 86 around a shaft 88, with screw blade 86 being designed to force grain through conveyor assembly 18 towards lower conveyor housing 30. Screw blade 86 is further designed to push grain in sump 24 past moisture sensor 58 to allow moisture sensor 58 to detect the moisture content of the grain. Conveyor 28 can be rotated by way of a conveyor motor 90 operatively connected to conveyor 28. Sump 24 can further include a housing panel 92 to partially contain material within sump 24.

FIG. 7 illustrates a side perspective view of a grain cart 100 in accordance with another embodiment. For convenience, elements of cart 100 that are functionally similar to cart 10 described herein will be identified by the same reference numbers. It is appreciated that these elements may not be identical to each other and that certain modifications to each element may be made to correspond to the applicable embodiment. For example, cart 100 can include a frame 12, a hopper 16, and a conveyor assembly 18. Cart 100 further includes a tread assembly 102 but can include one or more wheels similar to wheels 14 of cart 10. Cart 100 is positioned with its conveyor assembly 18 in an unfolded/extended position.

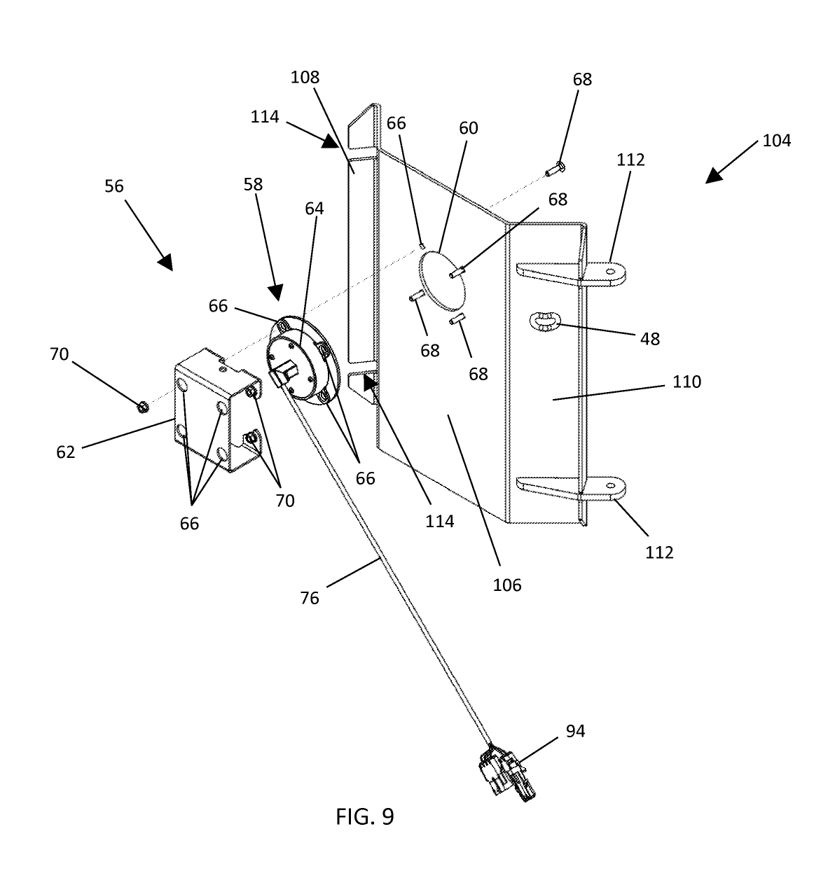

FIGS. 8 and 9 illustrate a door assembly 104 for use with cart 100. In particular, FIG. 8 illustrates an enlarged cut-away view of section B of cart 100 that shows details of door assembly 104, and FIG. 9 illustrates an exploded view of door assembly 104. Door assembly 104 includes a door panel 106 having multiple bent portions 108 and 110 which can correspond to the curvature of lower conveyor housing 30. Door panel 106 includes an opening 60 for receiving a moisture sensor 58. Embodiments of moisture sensor assembly 56 for use with door assembly 104 can be the same or similar to the moisture sensor assembly 56 described above with respect to door assembly 42. For example, moisture sensor assembly 56 for use with door assembly 104 can be secured to door panel 106 using similar attachment structures as moisture sensor assembly 56 described above with respect to FIG. 3, such as a bracket 62, bolt holes 66, and bolts 68.

Door assembly 104 is rotatably secured to cart 100 at a first end via one or more hinges 112. Door assembly 104 can be secured at a second end to cart 100 via one or more latch openings 114 that are sized to receive corresponding latches 134 (see, e.g., FIG. 8) fixed to cart 100.

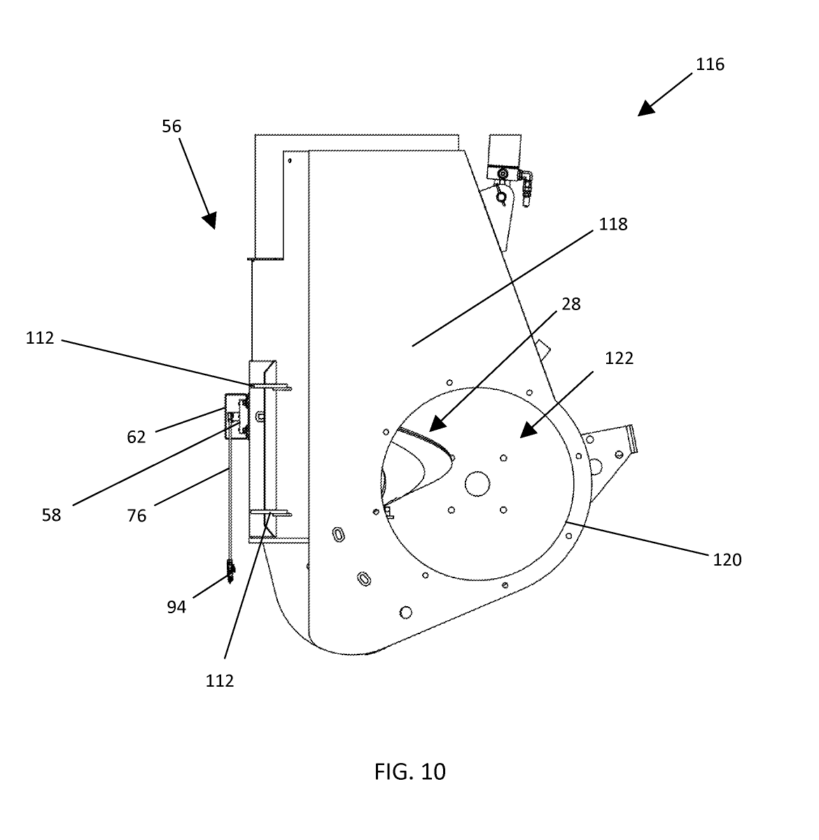

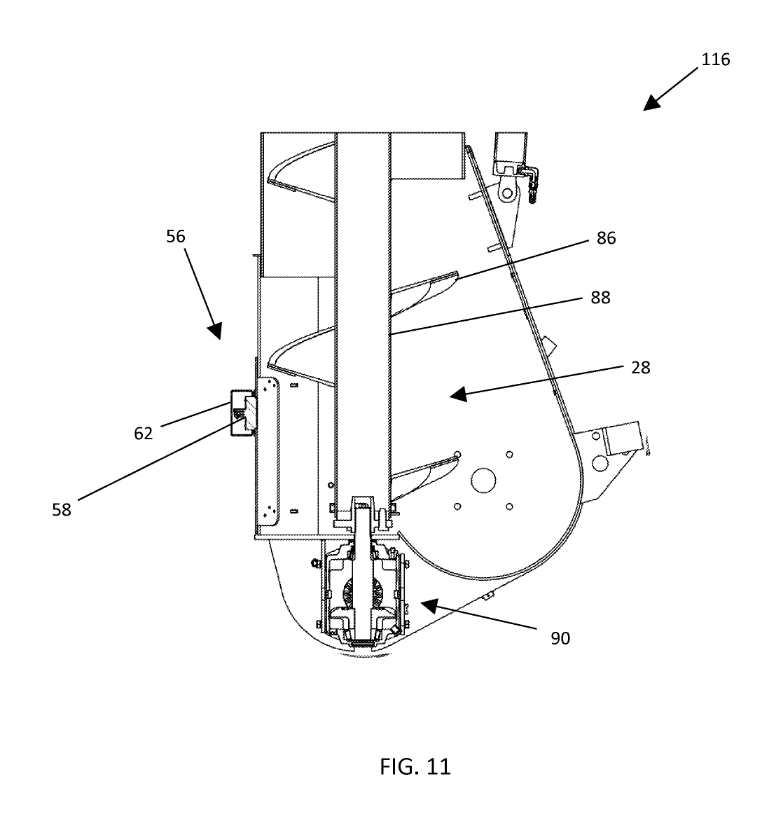

FIGS. 10 and 11 illustrate views of a portion of cart 100 with certain elements removed to show details of sump 116. In particular, FIG. 10 illustrates a top view of sump 116 and FIG. 11 illustrates a cross-sectional view of sump 116 along a rotational axis of conveyor 28. Similar to sump 24, sump 116 can include a housing panel 118 for assisting with containing harvested material within sump 116. Housing panel 118 can include an opening 120 formed therein to expose a receiving area 122 of sump 116. Similar to sump 24, sump 116 can include a conveyor 28 in the form of an auger having a helical screw blade 86 for conveying harvested material through sump 116.

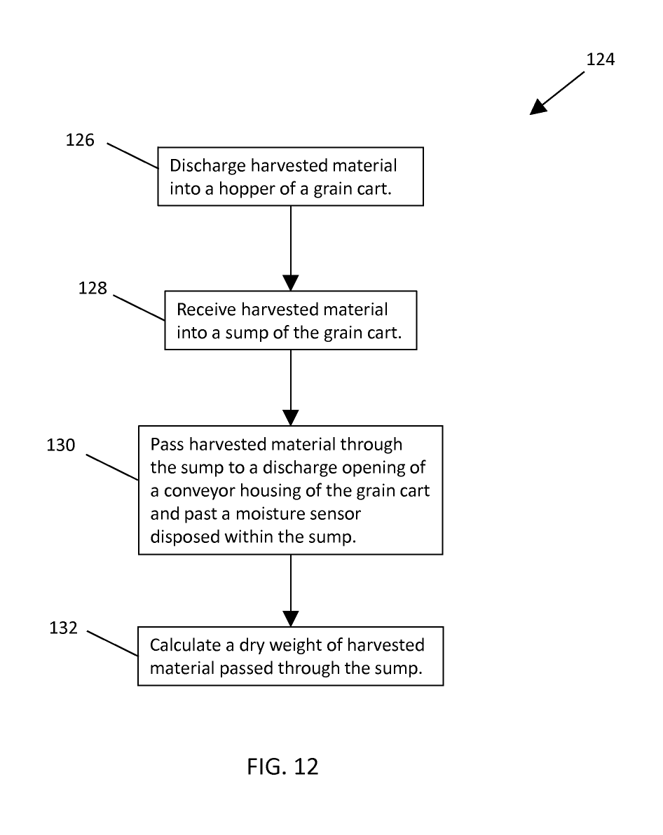

FIG. 12 illustrates a method 124 for calculating a dry weight of harvested material discharged from a grain cart. For convenience, this description of method 124 refers to elements of cart 10. It is appreciated, however, that other embodiments of carts described herein, such as for example cart 100, can be used with method 124. Method 124 can include the step 126 of discharging harvested material from a harvest vehicle into hopper 16 of cart 10.

Method 124 can further include the step 128 of receiving harvested material in sump 24 connected to an opening of hopper 16. Method 124 can further include the step 130 of passing the harvested material through sump 24 to a discharge opening 26 of a conveyor housing (formed by lower conveyor housing 30 and upper conveyor housing 32) so as to discharge the harvested material from cart 10. Step 130 can, for example, include passing the harvested material past moisture sensor 58 disposed within sump 24 that is designed to detect the moisture content of harvested material passed through sump 24. The moisture sensor 58 may read the moisture content of the harvested material during the entire unload cycle, and the average moisture content of the harvested material unloaded may be calculated. This is beneficial when receiving harvested material from multiple harvesters operating in different areas of a field and the harvested material in the hopper represents a wide sample of the grain moisture in the field.

Method 124 can further include the step 132 of calculating a dry weight of harvested material received within hopper 16 based on the moisture content of the harvested material passed through sump 24 and the weight of the harvested material received in hopper 16. Step 132 can, for example, be based on a predetermined crop standard moisture for the harvested material within hopper 16. For example, if the harvested material within hopper 16 is corn, the crop standard moisture can be approximately 15.5%. In some embodiments, a dry weight of harvested material within hopper 16 is calculated using the following formula: (weight of harvested material within the hopper)×(100−sensed moisture percentage of harvested material passed through the sump)/(100−crop standard moisture). The sensed moisture percentage of harvested material passed through sump 24 can, for example, be based on readings from moisture sensor 58. In some embodiments, method 124 can include a further step of an operator selecting a type of harvested material (e.g., corn) used for the calculation using an input device, such as a handheld controller operatively connected to cart 10.

While various embodiments/variations of the present invention have been described above, it should be understood that they have been presented by way of example only, and not limitation. Thus, the breadth and scope of the present invention should not be limited by any of the above-described exemplary embodiments. Further, unless stated, none of the above embodiments are mutually exclusive. Thus, the present invention may include any combinations and/or integrations of the features of the various embodiments.

Additionally, while the invention has been particularly taught and described with reference to certain preferred embodiments, those versed in the art will appreciate that modifications in form and detail may be made without departing from the spirit and scope of the invention.

All numbers in this description and figures indicating amounts, ratios of materials, physical properties of materials, and/or use are to be understood as modified by the word “about,” except as otherwise explicitly indicated. Dimensions shown in the figures are designated in inches. The choice of materials for the parts described herein can be informed by the requirements of mechanical properties, temperature sensitivity, moldability properties, or any other factor apparent to a person having ordinary skill in the art. For example, one or more of the parts described herein (or a portion of one of the parts) can be made from suitable metals, alloys, plastics, and/or other suitable materials. For example, one or more of the conveyors described herein can be in the form of steel augers.

In the preceding specification, various preferred embodiments have been described with reference to the accompanying drawings. It will, however, be evident that various modifications and changes may be made thereto, and additional embodiments may be implemented, without departing from the broader scope of the invention. The specification and drawings are accordingly to be regarded in an illustrative rather than restrictive sense. It is also appreciated that the steps of the various methods described herein may be performed in any suitable order.

It will also be appreciated that the above example components and operations are illustrative only, and that an embodiment of the present application may have fewer or more components or operations than those illustrated above, and have operations arranged in an order different than that illustrated above.

1. A grain cart comprising:
a hopper for transporting harvested material, the hopper including:
a first opening for receiving harvested material discharged from a harvesting vehicle;
a second opening for unloading harvested material from the hopper; and
a conveyor assembly operatively connected to the second opening so as to allow harvested material to be unloaded from the hopper using the conveyor assembly, the conveyor assembly including:
a sump operatively connected to the second opening of the hopper so as to receive harvested material from the hopper;
a conveyor housing including a discharge opening for discharging harvested material from the conveyor assembly; and
a conveyor operatively connected to the sump so as to move harvested material from the sump to the discharge opening of the conveyor housing,
wherein the sump includes a moisture sensor designed to detect the moisture content of harvested material passed through the sump.

2. The grain cart of claim 1, wherein the conveyor assembly is in the form of an auger assembly that includes:
an auger at least partially located in the sump, the auger including a helical screw blade designed to force harvested material through the conveyor assembly towards the discharge opening,
wherein the screw blade is further designed to push harvested material in the sump past the moisture sensor to allow the moisture sensor to detect the moisture content of the harvested material.

3. The grain cart of claim 1, wherein the conveyor assembly further includes:
a door assembly operatively connected to the sump so as to allow an operator to open the door assembly and access an interior cavity of the sump; and
wherein the moisture sensor is disposed in the door assembly so as to detect the moisture content of harvested material passed through the sump.

4. The grain cart of claim 3, wherein the door assembly includes:
a door panel having an opening formed therein,
wherein the moisture sensor is aligned with the opening of the door panel such that harvested material passing through the sump is passed by the moisture sensor so as to allow the moisture sensor to detect the moisture content of the harvested material.

5. The grain cart of claim 3, wherein the moisture sensor is recessed from an interior surface of the door panel facing the sump.

6. The grain cart of claim 3, wherein the moisture sensor has a sensing area with a diameter ranging from approximately 2.5 to approximately 3 inches.

7. The grain cart of claim 1, further comprising:
a scale operatively connected to the hopper so as to measure the weight of the harvested material received within the hopper.

8. The grain cart of claim 1, wherein the first opening of the hopper is located at the top of the hopper.

9. The grain cart of claim 1, wherein the second opening of the hopper is located at the bottom of the hopper.

10. The grain cart of claim 1, wherein the grain cart is configured to transport corn.

11. The grain cart of claim 1, wherein the harvesting vehicle is a combine harvester.

12. The grain cart of claim 1, wherein the conveyor assembly is configured to unload harvested material from the hopper to a semi-trailer.

13. The grain cart of claim 1, further comprising:
a processor operatively connected to the moisture sensor so as to receive sensor readings from the sensor,
wherein the processor is configured to calculate a dry weight of harvested material received within the hopper based on the moisture content of harvested material passed through the sump and a weight of the harvested material received in the hopper.

14. The grain cart of claim 13, wherein the processor is configured to calculate a dry weight of harvested material received within the hopper based on a predetermined crop standard moisture for harvested material within the hopper.

15. The grain cart of claim 13, further comprising:
an input device operatively connected to the processor that allows an operator to select a type of harvested material received in the hopper to allow the processor to automatically calculate the dry weight of harvested material received within the hopper.

16. The grain cart of claim 1, wherein the moisture sensor is positioned at a bottom of the sump such that the harvested material received in the sump moves towards the moisture sensor due to gravity.

17. A method for calculating a dry weight of harvested material discharged from a grain cart, the method comprising:
discharging harvested material from a harvest vehicle into a hopper of the grain cart;
receiving harvested material in a sump connected to an opening of the hopper;
passing the harvested material through the sump to a discharge opening of a conveyor housing so as to discharge harvested material from the grain cart, wherein the step of passing the harvested material through the sump includes passing the harvested material past a moisture sensor disposed within the sump that is designed to detect the moisture content of harvested material passed through the sump; and
calculating a dry weight of harvested material received within the hopper based on the moisture content of harvested material passed through the sump and the weight of the harvested material received in the hopper.

18. The method of claim 17, wherein calculating a dry weight of harvested material received within the hopper is further based on a predetermined crop standard moisture for the harvested material within the hopper.

19. The method of claim 17, wherein a dry weight of harvested material within the hopper is calculated using the following formula: (weight of harvested material within the hopper)×(100−sensed moisture percentage of harvested material passed through the sump)/(100−crop standard moisture),
wherein the sensed moisture percentage of harvested material passed through the sump is based on readings from the moisture sensor.

20. The method of claim 17, wherein an operator selects a harvested material used for the calculation using a handheld controller.

21. The method of claim 17, wherein the harvested material within the hopper is corn and the crop standard moisture is approximately 15.5%.
